(19) United States
(12) Patent Application Publication
(10) Pub. No.: US 2026/0157648 A1
(43) Pub. Date: Jun. 11, 2026

(54) METHOD AND SYSTEM FOR PREDICTING A LIMIT TO A SUBJECT'S AUTOREGULATION RANGE

(71) Applicant: Becton, Dickinson and Company, Franklin Lakes, NJ (US)

(72) Inventors: Anusha Alathur Rangarajan, Irvine, CA (US); Antonio Albanese, Irvine, CA (US); Zhongping Jian, Irvine, CA (US); Paul B. Benni, Acton, MA (US)

(21) Appl. No.: 19/101,484

(22) PCT Filed: Aug. 3, 2023
(86) PCT No.: PCT/US2023/029402

§ 371 Date: Feb. 5, 2025

Related U.S. Application Data
(60) Provisional application No. 63/370,633, filed on Aug. 5, 2022.

(51) Int. Cl. A61B 5/026; A61B 5/1455

(52) U.S. Cl. CPC A61B 5/0261; A61B 5/14551

(57) ABSTRACT

A method and system for predicting a limit to a subject's autoregulation range is provided. The method includes: a) determining blood flow values representative of a subject's blood flow in an autoregulation range of the subject during a period of time; b) determining blood pressure values representative of the subject's blood pressure in the autoregulation range of the subject during the period of time; c) determining a mathematical function that represents the blood flow values versus the blood pressure values within the autoregulation range; d) using the mathematical function to produce a representation of the blood flow values versus the blood pressure values outside of the autoregulation range; and e) predicting a limit to the autoregulation range using the representation of the blood flow values versus the blood pressure values outside of the autoregulation range.

### BACKGROUND OF THE INVENTION

### 1. Technical Field

The present disclosure relates to medical systems and methods in general, and to medical systems and methods for predicting a limit to a subject's autoregulation range in particular.

### 2. Background Information

Autoregulation is a process in mammals that aims to maintain adequate and stable (e.g., “constant”) blood flow to organs (e.g., brain, heart, kidneys, etc.) for a range of perfusion pressures. Different organs display varying degrees of autoregulatory behavior. The renal, cerebral, and coronary circulations typically show excellent autoregulation, whereas skeletal muscle and splanchnic circulations show moderate autoregulation. The cutaneous circulation shows little or no autoregulatory capacity.

Cerebral autoregulation may be defined as the maintenance of constant cerebral blood flow (CBF) despite changes in cerebral perfusion pressure (CPP), where CPP is equivalent to mean arterial pressure (MAP) minus intracranial pressure (ICP). To facilitate the discussion herein, CPP and MAP will be discussed herein in terms of MAP unless otherwise stated. Cerebral autoregulation plays an important protective role against the danger of cerebral hypoxia/ischemia at low MAPs and the risk of brain edema at higher MAPs. Indeed, in normotensive humans, cerebral blood flow remains relatively but not absolutely constant over a range of perfusion pressures, which range is commonly referred to as the autoregulation range (sometimes referred to as autoregulation plateau). The lower endpoint of the autoregulation range is typically referred to as the lower limit of autoregulation (LLA) and the upper endpoint of the autoregulation range is typically referred to as the upper limit of autoregulation (ULA). FIG. 1 is a diagrammatic graph of CBF vs CPP, illustrating an autoregulation curve having an autoregulation range defined by an LLA and a ULA. As stated above, in the region outside of the LLA a subject may be in danger of cerebral hypoxia/ischemia at low CPPs/MAPs and in the region outside of the ULA a subject may be in danger of brain edema at higher CPPs/MAPs. Once the limits of autoregulation (i.e., LLA, ULA) are reached, cerebral blood flow increases (above ULA) or decreases (below LLA) passively with concomitant increases or decreases in perfusion pressure. It should be noted that the LLA and the ULA are not “hard points”, but rather are points at which the flow/pressure relationship maintained by autoregulation begins to change.

As will be discussed herein, LLAs and ULAs can vary substantially from subject to subject, varying as a function of factors such as a subject's overall health, dietary factors, arterial blood carbon dioxide partial pressures levels (hypercapnia, hypocapnia, normocapnia), and can vary over time for specific subjects, to name a few. Historically, a subject's LLA or ULA may be determined by monitoring a subject's CBF and CPP and evaluating the collected data for changes (e.g., inflection points in the curve) to determine the extents of the autoregulation range. A collected data approach like this, however, requires the collection of data in the region outside of the LLA and/or outside of the ULA; i.e., regions where a subject may be subject to ischemia or edema.

What is needed is a system and method for monitoring autoregulation that permits a prediction of the LLA before MAP reaches the LLA and/or permits a prediction of the ULA before the MAP reaches the ULA.

### SUMMARY

According to an aspect of the present disclosure, a method for predicting a limit to a subject's autoregulation range is provided that includes: a) determining blood flow values representative of a subject's blood flow in an autoregulation range of the subject during a period of time; b) determining blood pressure values representative of the subject's blood pressure in the autoregulation range of the subject during the period of time; c) determining a mathematical function that represents the blood flow values versus the blood pressure values within the autoregulation range; d) using the mathematical function to produce a representation of the blood flow values versus the blood pressure values outside of the autoregulation range; and e) predicting a limit to the autoregulation range using the representation of the blood flow values versus the blood pressure values outside of the autoregulation range.

In any of the aspects of embodiments described above and herein, the predicted limit is at least one of a lower limit of autoregulation or an upper limit of autoregulation.

In any of the aspects of embodiments described above and herein, the step of determining blood flow values representative of the subject's blood flow in the autoregulation range of the subject during the period of time may be performed non-invasively, and may be performed using a near infrared spectroscopy (NIRS) oximeter.

In any of the aspects of embodiments described above and herein, the step of determining blood flow values representative of the subject's blood flow in the autoregulation range of the subject during the period of time may include determining a cerebral blood flow of the subject.

In any of the aspects of embodiments described above and herein, the step of determining blood pressure values representative of the subject's blood pressure in the autoregulation range of the subject during the period of time may include determining mean arterial blood pressure (MAP) values of the subject or a parameter derived from the MAP values.

In any of the aspects of embodiments described above and herein, the step of determining the mathematical function may include fitting a third degree polynomial equation to the blood flow values versus the blood pressure values within the subject's autoregulation range, and the step of using the mathematical function to produce a representation of the blood flow values versus the blood pressure values outside of the autoregulation range may include extrapolating the third degree polynomial equation to represent the blood flow values versus the blood pressure values outside of the autoregulation range.

In any of the aspects of embodiments described above and herein, the step of determining the mathematical function may include fitting one or more sigmoid functions to the blood flow values versus the blood pressure values within the subject's autoregulation range.

In any of the aspects of embodiments described above and herein, the step of determining blood flow values representative of the subject's blood flow in the autoregulation range of the subject may be performed continuously during the period of time, and the step of determining blood pressure values representative of the subject's blood pressure flow in the autoregulation range of the subject may be performed continuously during the period of time, and the step of predicting the limit to the autoregulation range using the representation of the blood flow values versus the blood pressure values outside of the autoregulation range may be performed in real time.

According to another aspect of the present disclosure, a system configured to determine a limit to a subject's autoregulation range is provided that includes a blood flow measurement device, a blood pressure sensing device, and a system controller. The blood flow measurement device is configured to determine blood flow values representative of a subject's blood flow in an autoregulation range of the subject during a period of time and to produce first signals representative of the blood flow values. The blood pressure sensing device is configured to determine blood pressure values representative of the subject's blood pressure in the autoregulation range of the subject during the period of time and to produce second signals representative of the blood flow values. The system controller is in communication with the blood flow measurement device and the blood pressure sensing device. The system controller includes at least one processor and a memory device configured to store instructions, the stored instructions when executed cause the controller to: a) control the blood flow measurement device to determine the blood flow values representative of the subject's blood flow in the autoregulation range of the subject during the period of time and to produce the first signals representative of the blood flow values; b) control the blood pressure sensing device to determine the blood pressure values representative of the subject's blood pressure in the autoregulation range of the subject during the period of time and to produce the second signals representative of the blood pressure values; c) determine a mathematical function that represents the blood flow values versus the blood pressure values within the autoregulation range using the first signals and the second signals; d) produce a representation of the blood flow values versus the blood pressure values outside of the autoregulation range using the mathematical function; and e) predict a limit to the autoregulation range using the representation of the blood flow values versus the blood pressure values outside of the autoregulation range.

In any of the aspects of embodiments described above and herein, the blood flow measurement device may be a NIRS oximeter configured to determine blood flow values non-invasively.

In any of the aspects of embodiments described above and herein, the blood pressure sensing device may be configured to determine mean arterial blood pressure (MAP) values of the subject or a parameter derived from the MAP values.

In any of the aspects of embodiments described above and herein, the mathematical function may be a third degree polynomial equation fit to the blood flow values versus the blood pressure values within the subject's autoregulation range, and the instructions that when executed may cause the controller to extrapolate the third degree polynomial equation to represent the blood flow values versus the blood pressure values outside of the autoregulation range.

In any of the aspects of embodiments described above and herein, the mathematical function may be a sigmoid function fit to the blood flow values versus the blood pressure values within the subject's autoregulation range, and the instructions that when executed may cause the controller to extrapolate the sigmoid function to represent the blood flow values versus the blood pressure values outside of the autoregulation range.

In any of the aspects of embodiments described above and herein, the instructions that when executed may cause the controller to: a) control the blood flow measurement device to continuously determine the blood flow values representative of the subject's blood flow in the autoregulation range during the period of time; b) control the blood pressure measurement device to continuously determine the blood pressure values representative of the subject's blood pressure flow in the autoregulation range during the period of time; and c) predict the limit to the autoregulation range using the representation of the blood flow values versus the blood pressure values outside of the autoregulation range in real time.

According to another aspect of the present disclosure, a non-transitory computer readable medium storing executable instructions is provided. The executable instructions are configured to, when executed, cause at least one processor to: a) control a blood flow measurement device to determine blood flow values representative of the subject's blood flow in an autoregulation range of the subject during a period of time and to produce first signals representative of the blood flow values; b) control a blood pressure sensing device to determine blood pressure values representative of the subject's blood pressure in the autoregulation range of the subject during the period of time and to produce second signals representative of the blood pressure values; c) determine a mathematical function that represents the blood flow values versus the blood pressure values within the autoregulation range using the first signals and the second signals; d) produce a representation of the blood flow values versus the blood pressure values outside of the autoregulation range using the mathematical function; and e) predict a limit to the autoregulation range using the representation of the blood flow values versus the blood pressure values outside of the autoregulation range.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic graph of CBF versus CPP illustrating an autoregulation curve, an LLA, a ULA and the autoregulation range/plateau.

FIG. 2 is a diagrammatic representation of a present disclosure system embodiment.

FIG. 3 is a diagrammatic representation of a present disclosure system embodiment.

FIG. 4 is a diagrammatic representation of an exemplary frequency domain method.

FIG. 5 is a diagrammatic graph of AR Index versus MAP illustrating data points collected in the autoregulation range.

FIG. 6 is a diagrammatic illustration of a third degree polynomial equation for a function ƒ(x) relative to an X-axis.

FIG. 7 illustrates a sigmoid function.

FIG. 8 is a graph illustrating an example of an elbow point methodology.

FIG. 9A is a graph of CBF versus MAP illustrating data points collected in an animal study, a third degree polynomial curve fit to the data points in the autoregulation range and beyond, and an LLA determined from the data points.

FIG. 9B is a graph of CBF versus MAP illustrating data points shown in FIG. 9A, showing only those data points collected in the autoregulation range with a third degree polynomial curve fit to the data points.

FIG. 9C is a graph of CBF versus MAP illustrating data points shown in FIG. 9A, showing only those data points collected in the autoregulation range with a third degree polynomial curve fit to the data points, now extrapolated beyond the autoregulation range.

FIG. 10A is a graph of CBF versus MAP illustrating data points collected in an animal study, a third degree polynomial curve fit to the data points in the autoregulation range and beyond, and an LLA determined from the data points.

FIG. 10B is a graph of CBF versus MAP illustrating data points shown in FIG. 10A, showing only those data points collected in the autoregulation range with a third degree polynomial curve fit to the data points.

FIG. 10C is a graph of CBF versus MAP illustrating data points shown in FIG. 10A, showing only those data points collected in the autoregulation range with a third degree polynomial curve fit to the data points, now extrapolated beyond the autoregulation range.

FIG. 11 is a diagrammatic graph of NIRS versus MAP, illustrating an autoregulation curve based on a sigmoidal curve fit to data points.

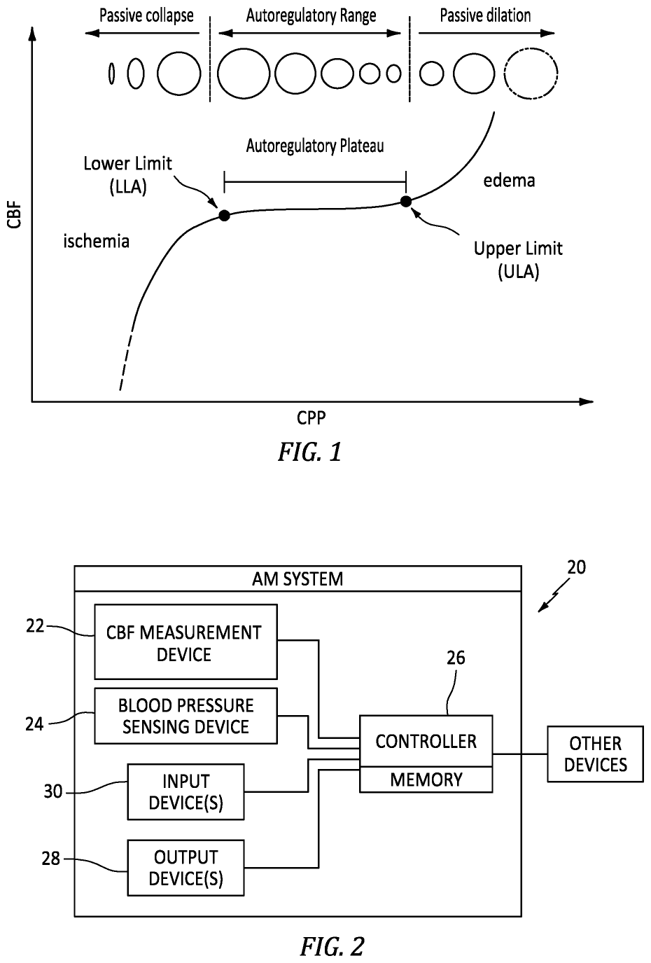

### DETAILED DESCRIPTION

The present disclosure is directed to a system 20 and method for monitoring autoregulation function, that permits a prediction of a lower limit of autoregulation (LLA) for a subject's autoregulation range (i.e., autoregulation plateau) and/or a prediction of an upper limit of autoregulation (ULA) for a subject's autoregulation range that does not require the collection of data in the region outside of the subject's autoregulation range (i.e., outside of the LLA and/or ULA), and a non-transitory computer-readable medium containing instructions for carrying out the present disclosure method.

As indicated above, the autoregulation process in mammals aims to maintain adequate and stable (e.g., “constant”) blood flow to organs (e.g., brain, heart, kidneys, etc.) for a range of perfusion pressures. To facilitate the description herein, the present disclosure is described in terms of cerebral autoregulation. The present disclosure is not limited to use with cerebral autoregulation and may be used for autoregulation function determinations for other organs.

A determination of a subject's autoregulation state utilizes a measurement of blood flow and a measurement of blood pressure. In terms of cerebral autoregulation, the autoregulation state determination utilizes a measurement of cerebral blood flow (CBF) or a surrogate thereof and a measurement of cerebral perfusion pressure (CPP) or mean arterial pressure (MAP). The term “surrogate” as used with CBF herein refers to a physiologic parameter that corresponds with CBF, or that is analogous to CBF, or from which CBF may be determined, or the like. To facilitate the description herein, the term “CBF” as used herein is intended to mean cerebral blood flow and/or a surrogate of cerebral blood flow unless otherwise indicated. CBF may be measured in a variety of different ways. CBF measurement devices 22 that invasively measure CBF may use techniques such as the flow laser doppler flow meter. CBF measurement devices 22 that directly measure CBF or indirectly measure CBF (e.g., via a surrogate) may use non-invasive techniques such as near-infrared spectroscopy (NIRS), transcranial Doppler ultrasound imaging, phase-contrast MRI, and arterial spin labeling MRI (ASL-MRI). The present disclosure is not limited to any particular methodology for measuring CBF or any CBF measuring device 22 configuration. Non-limiting examples provided herein include a CBF measuring device 22 in the form of a NIRS tissue oximeter that is operable to produce a blood parameter measurement (e.g., a NIRS index such as StO2, rTHb, differential changes in O2Hb and HHb, etc.) that is a surrogate of CBF.

MAP may be measured in a variety of different ways. Non-limiting examples of a blood pressure sensing device (“BP sensing device 24”) that may be used to measure a subject's MAP include an arterial catheter line, a continuous non-invasive blood pressure device, a pulse oximetry sensor, or the like. The present disclosure is not, however, limited to using any particular type of BP sensing device. The BP sensing device 24 may be configured to continuously produce a MAP measurement. The term “continuously” as used herein (regarding a BP sensing device 24) means that the BP sensing device 24 senses and collects subject data on a periodic basis during the monitoring time period, which periodic basis is sufficiently frequent that it may be considered to be clinically continuous. For example, some BP sensing devices 24 sample data every ten seconds or less and can be configured to sample data more frequently (e.g., every two seconds or less).

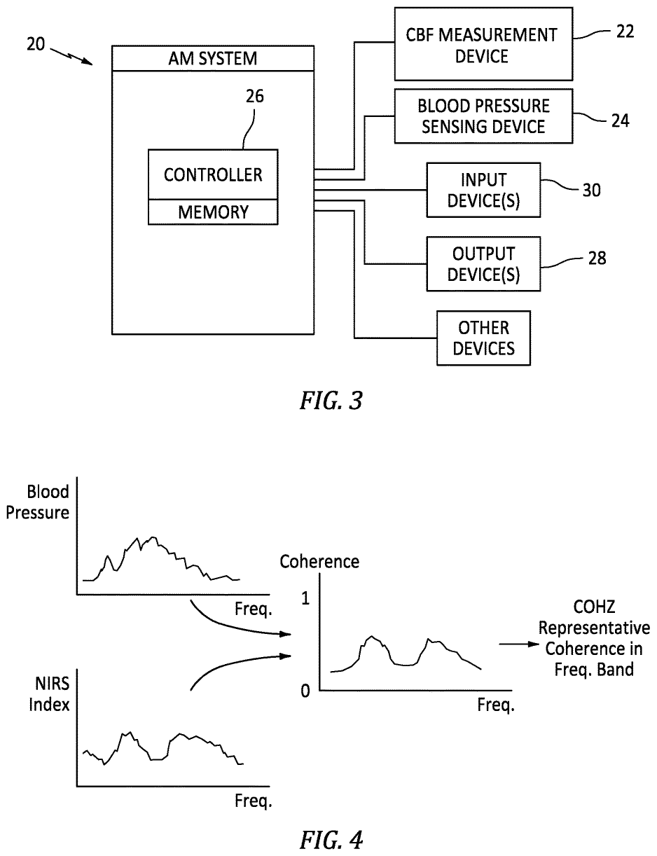

Non-limiting examples of a present disclosure system 20 are diagrammatically shown in FIGS. 2 and 3. The system 20 embodiment diagrammatically shown in FIG. 2 is configured to include system components (e.g., CBF measurement device 22, BP sensing device 24, system controller 26, etc.) integrated into a single system device. The system 20 embodiment diagrammatically shown in FIG. 3 is configured to include a system controller 26, and other system components (e.g., CBF measurement device 22, BP sensing device 24, system controller 26, etc.) that are independently configured and in communication with (e.g., receive signal data from and/or send signal data to) the system controller 26. In other words, in a system 20 embodiments such as that shown in FIG. 3, the system 20 may be configured to communicate with a BP sensing device 24 capable of functioning independently of the system 20, a CBF measurement device capable of functioning independently of the system 20, etc. In other embodiments, the system 20 may include some combination of these components in integral and independent form. In those embodiments wherein one or more of the aforesaid components is independently configured, that independent component may be in communication with the system controller 26 in any manner; e.g., hardwire, wireless, etc.

The system controller 26 may include any type of computing device, computational circuit, or any type of process or processing circuit capable of executing a series of instructions that are stored in memory. The system controller 26 may include multiple processors and/or multicore CPUs and may include any type of processor, such as a microprocessor, digital signal processor, co-processors, a micro-controller, a microcomputer, a central processing unit, a field programmable gate array, a programmable logic device, a state machine, logic circuitry, analog circuitry, digital circuitry, etc., and any combination thereof. For example, in those embodiments of the system 20 described above that include multiple components integral with the system 20 (e.g., a BP sensing device 24, a CBF measurement device 22, etc.) integral with the system 20, the system controller 26 may include multiple processors; e.g., an independent processor dedicated to each respective component, any and all of which processors may be in communication with a central processor of the system 20 that coordinates functionality of the system 20. The instructions stored in memory may represent one or more algorithms for controlling the system 20, and the stored instructions are not limited to any particular form (e.g., program files, system data, buffers, drivers, utilities, system programs, etc.) provided they can be executed by the system controller 26. The instructions are configured to perform the methods and functions described herein.

The system controller 26 may be configured (e.g., via electrical circuitry) to process various received signals (received from integral or independent components) and may be configured to produce certain signals to the same; e.g., signals configured to control one or more components within the system 20. Alternatively, the system 20 may be configured such that signals from a respective component are sent to one or more intermediate processing devices, and the intermediate processing device may in turn provide processed signals or data to the system controller 26. The system controller 26 may be configured to execute stored instructions (e.g., algorithmic instructions) that cause the system 20 to perform steps or functions described herein, to produce data (e.g., measurements, etc.) relating to a subject's autoregulation function, to communicate, etc.

The memory may be a machine readable storage medium configured to store instructions that when executed by one or more processors, cause the one or more processors to perform or cause the performance of certain functions. The memory may be a single memory device or a plurality of memory devices. A memory device may be a non-transitory device and may include a storage area network, network attached storage, as well as a disk drive, a read-only memory, random access memory, volatile memory, non-volatile memory, static memory, dynamic memory, flash memory, cache memory, and/or any device that stores digital information. One skilled in the art will appreciate, based on a review of this disclosure, that the implementation of the system controller 26 may be achieved via the use of hardware, software, firmware, or any combination thereof.

In some embodiments, the system 20 may include one or more output devices 28 and one or more input devices 30. Non-limiting examples of an input device 30 include a keyboard, a touchpad, or other device wherein a user may input data, commands, or signal information, or a port configured for communication with an external input device via hardwire or wireless connection, etc. Non-limiting examples of an output device 28 include any type of display, printer, or other device configured to display or communicate information or data produced by the system 20. The system 20 may be configured for connection with an input device 30 or an output device 28 via a hardwire connection or a wireless connection.

Implementation of the techniques, blocks, steps, and means described herein may be done in various ways. For example, these techniques, blocks, steps, and means may be implemented in hardware, software, or a combination thereof. For a hardware implementation, processing devices configured to carry out the described functions and steps (e.g., by executing stored instructions) may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, and/or any combination thereof.

Embodiments of the present disclosure may be described herein as a process which is depicted in a flowchart, a flow diagram, a block diagram, etc. Although any one of these logical structures may describe the operations as a sequential process, many of the operations can be performed in parallel or concurrently, or in a rearranged order. A process may correspond to a method, a function, a procedure, a subroutine, a subprogram, etc.

As stated above, a determination of a subject's autoregulation state utilizes a measurement of blood flow (e.g., a measurement of CBF) and a measurement of blood pressure (e.g., MAP or CPP) and the present disclosure permits a determination of an LLA and/or a ULA for a subject's autoregulation range that does not require the collection of data in the region outside of the subject's autoregulation range (e.g., outside of the LLA and/or outside of the ULA). The present disclosure is not limited to using any particular type of CBF measurement device 22 or BP sensing device 24 to determine data points within a subject's autoregulation range.

In a first nonlimiting example, a present disclosure system utilizes a NIRS tissue oximeter as a CBF measurement device 22. U.S. Patent Publication 2020/038616 and/or PCT Patent Application No. PCT/US2022/027282 (collectively referred to hereinafter as a “NIRS AR system”) describe NIRS tissue oximetry systems that include a NIRS tissue oximeter configured to provide CBF data (e.g., indirectly via a physiologic parameter data that corresponds with CBF such as a NIRS index value (StO2), an AR index value, etc.) and autoregulation state data. Both of these patent applications are commonly assigned to the present applicant and both are hereby incorporated by reference in its respective entirety.

The NIRS tissue oximeter includes one or more sensors in communication with a controller portion. Each sensor includes one or more light sources (e.g., light emitting diodes, or “LEDs”) and one or more light detectors (e.g., photodiodes, etc.). The light sources are configured to emit light at different wavelengths of light, e.g., wavelengths of light in the red or near infrared range; 400-1000 nm. In some sensor embodiments, a sensor may be configured to include a light source(s), a near detector(s), and a far detector(s). The near detector is disposed closer to the light source than the far detectors. A non-limiting example of such a sensor is disclosed in U.S. Pat. No. 8,965,472. NIRS tissue oximeters may utilize one or more algorithms to determine a NIRS index value which may be used to determine autoregulation data, or the aforesaid algorithms may be used to determine CBF data which may be used to determine autoregulation data. U.S. Pat. Nos. 10,117,610; 9,913,601; 9,848,808; 9,456,773; 9,364,175; 9,923,943; 8,965,472; 8,788,004; 8,396,526; 8,078,250; 7,072,701; and 6,456,862 all describe non-limiting examples of NIRS tissue oximeters and respective algorithms that may be used with the present disclosure, and all are incorporated by reference in their respective entirety herein.

In some embodiments, a NIRS tissue oximeter may be utilized to continuously sense tissue to produce NIRS index data that may be directly or indirectly (e.g., processed to determine CBF data) used in an autoregulation determination. The term “continuously” as used herein (regarding a NIRS tissue oximeter) means that the NIRS tissue oximeter senses and collects subject data on a periodic basis during the monitoring time period, which periodic basis is sufficiently frequent that it may be considered to be clinically continuous. For example, some NIRS tissue oximeters sample data every ten seconds or less and can be configured to sample data more frequently (e.g., every two seconds or less).

The NIRS AR system utilizes real-time data collection of tissue oxygen saturation data that is used to produce NIRS index data/CBF data and MAP data to produce autoregulation function data. The NIRS AR system may be configured to produce autoregulation data using an algorithm based on a frequency domain methodology to produce a coherence (COHZ) analysis, or using an algorithm based on correlation/regression technique, or some combination thereof. FIG. 4 diagramatically depicts an exemplary frequency domain method that involves taking synchronous blood pressure and NIRS index values over a predetermined sampling window (e.g., period of time). In this exemplary frequency domain method, the blood pressure and NIRS index values are each transformed (e.g., via a Fourier transformation) from a time domain to a frequency domain (shown as respective plots of blood pressure versus frequency and NIRS index versus frequency) and the transformed data is further analyzed to determine the degree of coherence within a single band of frequencies (i.e., a single frequency band). In some embodiments, the same process may be utilized to determine the degree of coherence in a plurality of frequency bands for each physiologic parameter (i.e., NIRS index and BP), and a collective coherence value or a value representative thereof may be determined. In either instance, the coherence values may be expressed as an autoregulation representative value such as an AR Index value or a pressure passive index (PPI) value. The degree of coherence (e.g., the COHZ value, the AR index, or the PPI) may be expressed in terms of an arbitrarily assigned scale of zero to one hundred (e.g., 0-100), wherein the degree of coherence increases from zero to one hundred. A coherence value of one hundred represents a pressure passive condition as described above. Conversely, a coherence value that approaches zero indicates increasingly less relationship between the NIRS index and blood pressure parameter. Coherence values (e.g., COHZ, AR index, or PPI) determined over a period of time may be binned in blood pressure increments (e.g., every 5 mmHg) or in incremental blood pressure ranges (e.g., 0-20 mmHg, 20-25 mmHg, 25-30 mmHg, etc.).

In some embodiments of the present disclosure utilizing a NIRS AR system, the coherence determination process may be executed for a plurality of different NIRS indices (e.g., StO2, rTHb, differential changes in O2Hb and HHb, HbD, etc.). In an instance where one NIRS index is more sensitive to autoregulation function than another, performing the autoregulation function determination processes as described herein (e.g., within a single frequency band, or within a plurality of frequency bands) can provide additional sensitivity and/or faster identification of change in a subject's autoregulation function.

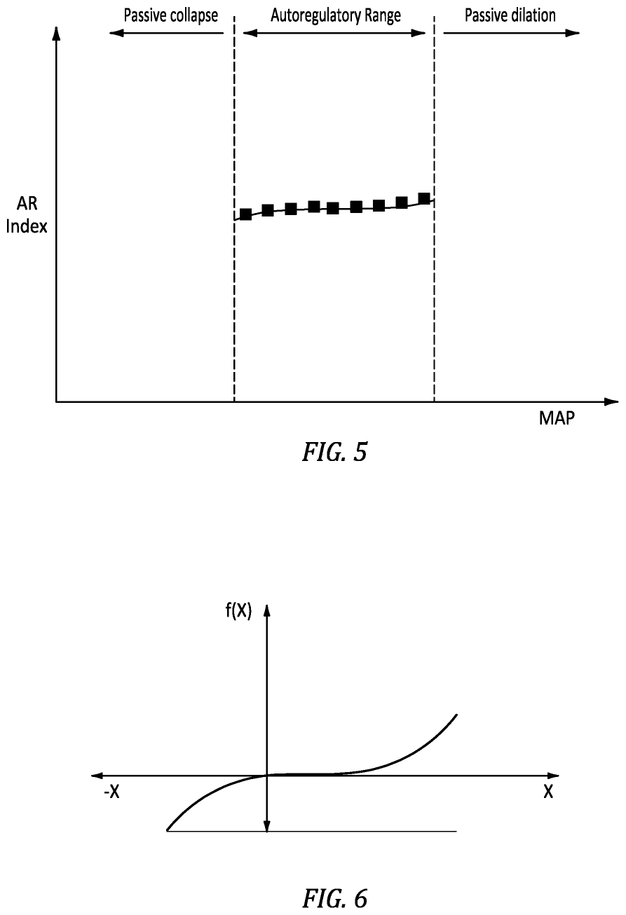

The degree of coherence (e.g., the COHZ value, the AR index, or the PPI) determined as a function of MAP may be used to produce an autoregulation curve. FIG. 5 illustrates a graph of AR index versus MAP data points within the autoregulation range. These data points—in the autoregulation range—represent determined data points based on physiologic parameter data sensed from a subject. As will be clear from the description below, the present disclosure obviates the need to collect autoregulation data (e.g., CBF, AR index, a NIRS index, MAP, etc.) under conditions below the LLA which may cause the subject to experience ischemia, and/or the need to collect autoregulation data under conditions above the ULA which may cause the subject to experience edema. A person of skill in the art will recognize the clinical benefits associated with avoiding a subject to conditions which may lead to or cause ischemia and/or edema.

To be clear, the example provided above illustrates how a NIRS tissue oximeter may be used with a BP sensing device 24 to determine such data points within a subject's autoregulation range. The non-invasive NIRS tissue oximetry and the methodologies described in U.S. Patent Publication 2020/038616 and/or PCT Patent Application No. PCT/US2022/027282 can provide distinct advantages in the determination of autoregulation data. The present disclosure is not limited, however, to the aforesaid non-invasive NIRS tissue oximetry and the methodologies. In other embodiments, other devices configured to measure blood flow and blood pressure in a different manner (examples described above) may be used to determine data points within a subject's autoregulation range.

The present disclosure process for predicting an LLA value and/or a ULA value includes determining a mathematical function (e.g., curve-fitting) that has a best fit to the CBF versus MAP data points within a subject's autoregulation range (which data points are based on sensed data) regardless of the method used to produce such data points. The curve-fitting process not only permits the CBF versus MAP data points within the autoregulation range to be characterized, but also permits the curve to be extrapolated beyond the data points (based on sensed data) within the autoregulation range; i.e., in the direction toward and past an LLA and/or in the direction toward and past a ULA. After an appropriate mathematical function that has a best fit to the CBF versus MAP data points within a subject's autoregulation range is determined, the mathematical function can be used to extrapolate beyond the autoregulation range, and an LLA and/or a ULA can be predicted based on the mathematical function.

A variety of different techniques are known for fitting mathematical functions to data points and the present disclosure is not limited to using any particular methodology for fitting a mathematical function to determined CBF versus MAP data points collected within a subject's autoregulation range. A first example of a methodology for fitting a mathematical function to determined CBF versus MAP data points that may be used involves fitting the data points to a third degree polynomial equation like the following:

$$f ( x ) = {ax}^{3} + {bx}^{2} + cx + d \qquad [ Eqn . 1 ]$$

where a, b, c, and d are coefficients that may be determined based on the determined CBF versus MAP data points. FIG. 6 graphically illustrates a third degree polynomial equation for a function ƒ(x) relative to an X-axis. For autoregulation range data fitting purposes, the function ƒ(x) may be restricted to a positive value.

Another nonlimiting example of a mathematical function that may be fit to CBF versus MAP data points collected within a subject's autoregulation range is a sigmoid function. Equation 2 below provides an example of a sigmoid function that may be used.

$$S ( x ) = \frac{1}{( 1 + e \hat{} \{ - x \} )} \qquad [ Eqn . 2 ]$$

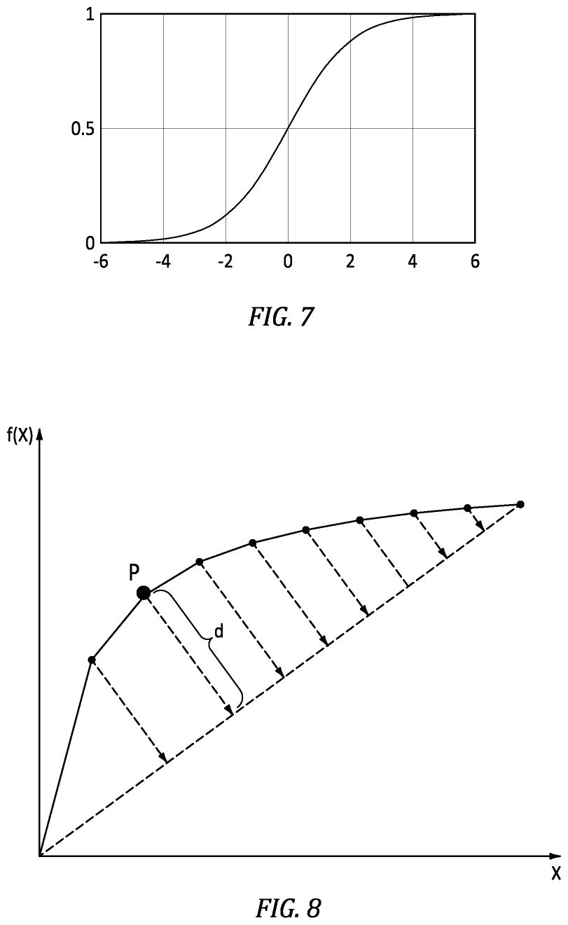

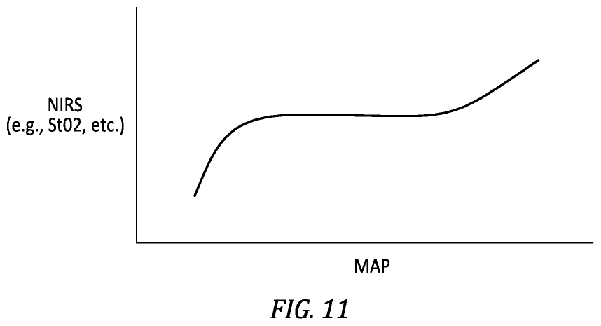

A sigmoid function is a mathematical function that has a characteristic “S”-shaped curve (sometime referred to as a “sigmoid curve”). An example of a sigmoid function that may be used with the present disclosure is graphically depicted in FIG. 7. As can be seen in FIG. 7, a sigmoidal curve has distinctive flat regions at two different values plus a curve region that is a transition zone between the two flat regions. FIG. 11 also illustrates a sigmoidal curve fit to NIRS versus MAP data points.

As stated above, third degree polynomial equations and sigmoid functions are non-limiting examples of mathematical functions that may be used to produce a best fit to CBF versus MAP data points within a subject's autoregulation range and the present disclosure is not limited thereto. In some embodiments, additional mathematical techniques such as non-linear regression may be used in combination to facilitate the process of fitting a mathematical function to the CBF versus MAP data points collected within a subject's autoregulation range.

Once a mathematical function (e.g., a third degree polynomial equation, a sigmoid function, etc.) is fit to the CBF versus MAP data points within a subject's autoregulation range and the function is extrapolated beyond the autoregulation range (i.e., in a direction toward and past an LLA and/or in a direction toward and past a ULA), a prediction of the LLA and/or the ULA can be made based on the respective extrapolated curve portion. In some embodiments, the determination of the LLA and/or the ULA may be made based on inflection points within the extrapolated curve. The inflection points may be identified in a variety of different ways. For example, an inflection point may be identified using slope values or based on a comparison of a slope value relative to a predetermined threshold value. As another example, an inflection point may be identified based on a rate of change value; e.g., a second derivative of the curve. Here again, the identification of an inflection point may be based on a rate of change value itself (e.g., a maximum rate of change) or based on a comparison of a rate of change value relative to a predetermined threshold value. As another example, an inflection point may be predicted as an “elbow point” on the extrapolated curve; e.g., for any the curve f(x), an “elbow point” methodology may be used to find a point “P” on the curve that has the maximum perpendicular distance “d” to a line joining the first and last points on the curve (e.g., see FIG. 8; Satopaa et al., “Finding a needle in a haystack: Detecting knee points in system behavior”; 2011 31^{st} International Conference on Distributed Computing Systems Workshops, IEEE 2011). As another example, an inflection point may be identified using slope and autoregulation data (e.g., CBF values, NIRS index values, AR Index values, etc.) being within a predetermined threshold that is based on empirical data.

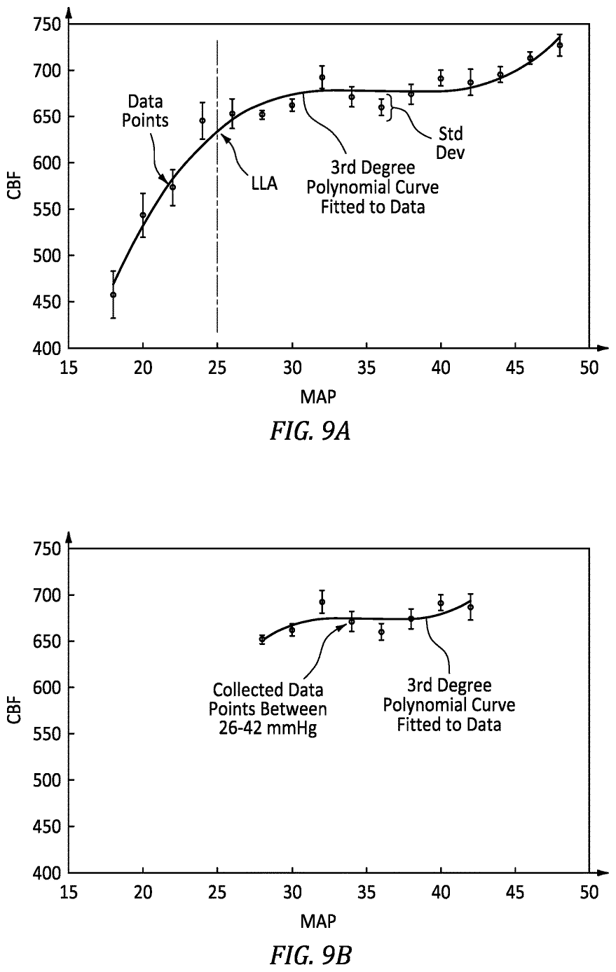

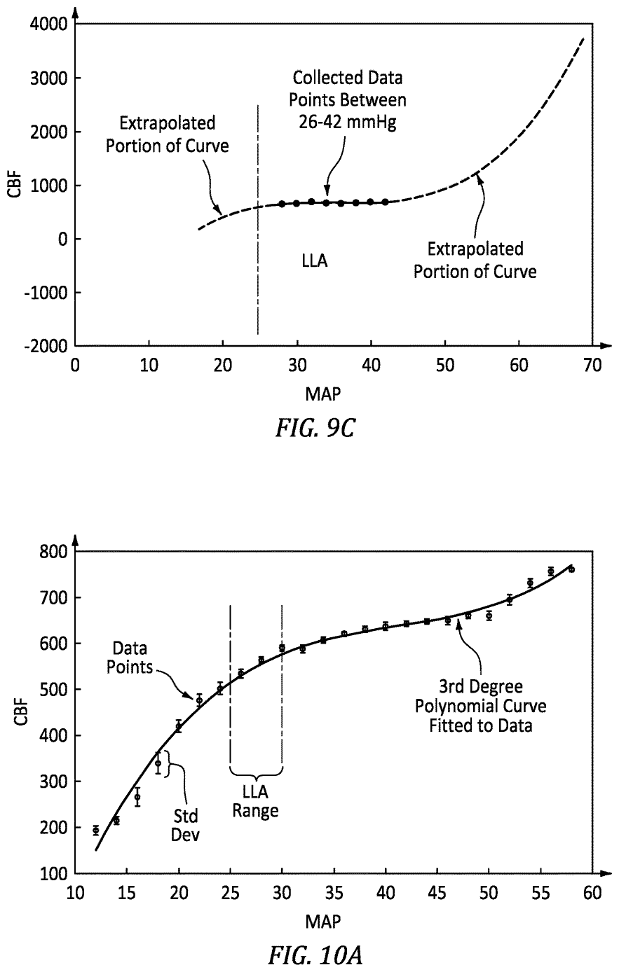

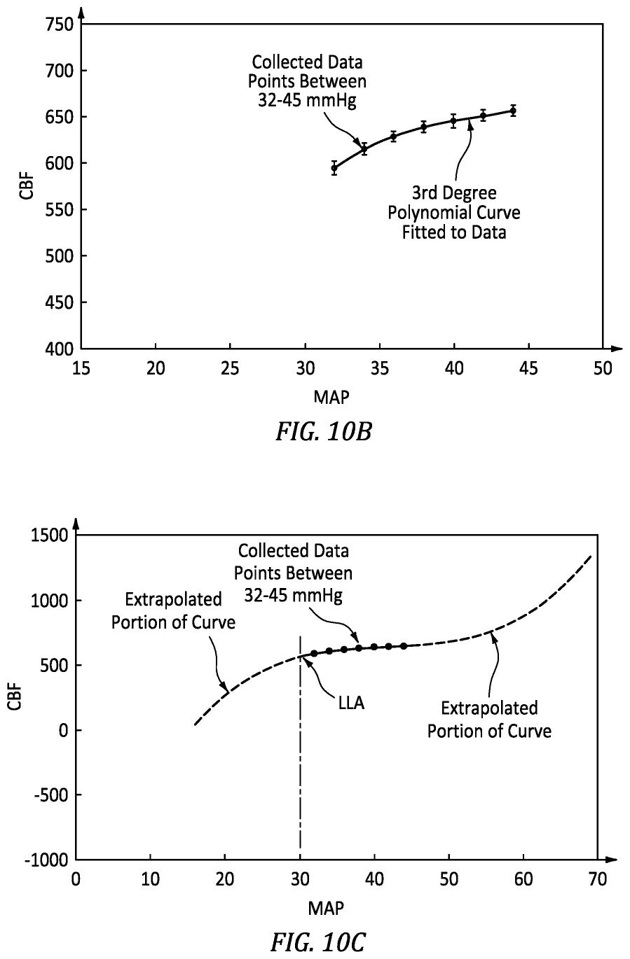

Specific examples illustrating the effectiveness of the present disclosure are provided in FIGS. 9A-9C and 10A-10C. As stated above, autoregulation data may be expressed as a function of blood flow (e.g., CBF) and MAP, or as a function of a surrogate of CBF and MAP. FIG. 11 illustrates a graph of NIRS (e.g., StO2) versus MAP that may be developed using NIRS oximetry techniques as described herein. FIG. 9A illustrates CBF versus MAP data points collected in an autoregulation study performed on a piglet under appropriate guidelines. The autoregulation study collected CBF versus MAP data points in the autoregulation range and beyond; e.g., in areas outside of the LLA and the ULA. The CBF versus MAP data points are shown in FIG. 9A, including vertical standard deviation ranges for each CBF versus MAP data point. FIG. 9A also illustrates a third degree polynomial curve fit to the collected CBF versus MAP data points. An LLA was determined (e.g., using a technique described above) at approximately 25 mmHg based on the CBF versus MAP data points (i.e., sensed data), including data points disposed in the autoregulation range and data points clearly outside of the autoregulation range and the LLA. FIG. 9B illustrates the collected CBF versus MAP data points between 26 mmHg and 42 mmHg (i.e., only in the autoregulation range) and the third degree polynomial curve fit to the collected CBF versus MAP data points between 26 mmHg and 42 mmHg. Visual inspection of the collected CBF versus MAP data points between 26 mmHg and 42 mmHg reveals no clear LLA. FIG. 9C illustrates the collected CBF versus MAP data points between 26 mmHg and 42 mmHg, the third degree polynomial curve fit to the collected CBF versus MAP data points between 26 mmHg and 42 mmHg, now including an extrapolated curve portion below 26 mmHg, and an extrapolated curve portion above 42 mmHg. The extrapolated curve portions are extrapolated from the curve fit to the collected CBF versus MAP data points between 26 mmHg and 42 mmHg and are not based on sensed data points outside of 26 mmHg and 42 mmHg. The extrapolated curve portion below 26 mmHg is used to predict the LLA; e.g., using a technique described above. The LLA predicted from the extrapolated curve portion below 26 mmHg (e.g., at approximately 25 mmHg) agrees with the LLA determined using the collected CBF versus MAP data points in the autoregulation range and in the area outside of the LLA. It is understood that the same result would have been arrived at if the CBF data from the study was replaced with NIRS index (e.g., StO2) developed within the study.

FIG. 10A illustrates CBF versus MAP data points collected in another autoregulation study performed on a piglet under appropriate guidelines. The autoregulation study collected CBF versus MAP data points in the autoregulation range and beyond; e.g., in areas outside of the LLA and the ULA. The CBF versus MAP data points (i.e., sensed data) are shown in FIG. 10A, including vertical standard deviation ranges for each CBF versus MAP data point. FIG. 10A also illustrates a third degree polynomial curve fit to the collected CBF versus MAP data points. An LLA was determined (e.g., using a technique described above) in between 25 mmHg and 30 mmHg based on the CBF versus MAP data points (i.e., sensed data), including data points disposed in the autoregulation range and data points clearly outside of the autoregulation range and the LLA. FIG. 10B illustrates the collected CBF versus MAP data points between 32 mmHg and 45 mmHg (i.e., only in the autoregulation range) and the third degree polynomial curve fit to the collected CBF versus MAP data points between 32 mmHg and 45 mmHg. Visual inspection of the collected CBF versus MAP data points between 32 mmHg and 45 mmHg reveals no clear LLA. FIG. 10C illustrates the collected CBF versus MAP data points (i.e., sensed data) between 32 mmHg and 45 mmHg, the third degree polynomial curve fit to the collected CBF versus MAP data points between 32 mmHg and 45 mmHg, now including an extrapolated curve portions below 32 mmHg and above 45 mmHg. The extrapolated curve portions are extrapolated from the curve fit to the collected CBF versus MAP data points between 32 mmHg and 45 mmHg and are not based on sensed data points outside of 32 mmHg and 45 mmHg. The extrapolated curve portion below 32 mmHg is used to predict the LLA; e.g., using a technique described above. The LLA predicted from the extrapolated curve portion below 32 mmHg (e.g., at approximately 30 mmHg) agrees with the LLA determined from the collected CBF versus MAP data points in the autoregulation range and in the area outside of the LLA. Here again, it is understood that the same result would have been arrived at if the CBF data from the study was replaced with NIRS index (e.g., StO2) developed within the study.

The examples shown in FIGS. 9A-C and 10A-10C illustrate well how an LLA can be predicted based on CBF versus MAP data points collected in the autoregulation range of a subject without the use of (or need for) CBF versus MAP data points collected outside of the subject's autoregulation range (i.e., outside of the LLA). The aforesaid methodology equally applies to determining/predicting a ULA.

As can be seen from the examples shown in FIGS. 9A-C and 10A-10C, the present disclosure permits a prediction of an autoregulation LLA and/or a ULA that obviates the need to collect data in the region outside of the subject's autoregulation range where the subject may be subject to ischemia or edema. A prediction of an LLA and/or a ULA before a subject experience's perfusion pressures outside the intact autoregulation range has significant clinical value. In addition as described above, processes for determining CBF and MAP can be performed on a continuous or near continuous basis. The present disclosure methodology for predicting an LLA and/or a ULA may also, therefore, predict a subject's LLA and/or ULA on a continuous or near continuous basis. The present disclosure therefore may provide a clinician with substantially real time LLA and/or ULA predictive information that may be clinically important in treating the subject.

As indicated above, the functionality described herein may be implemented, for example, in hardware, software tangibly embodied in a computer-readable medium, firmware, or any combination thereof. In some embodiments, at least a portion of the functionality described herein may be implemented in one or more computer programs. Each such computer program may be implemented in a computer program product tangibly embodied in non-transitory signals in a machine-readable storage device for execution by a computer processor. Method steps of the present disclosure may be performed by a computer processor executing a program tangibly embodied on a computer-readable medium to perform functions of the present disclosure by operating on input and generating output. Each computer program within the scope of the present claims below may be implemented in any programming language, such as assembly language, machine language, a high-level procedural programming language, or an object-oriented programming language. The programming language may, for example, be a compiled or interpreted programming language.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

The treatment techniques, methods, and steps described or suggested herein or in references incorporated herein may be performed on a living animal or on a non-living simulation, such as on a cadaver, cadaver heart, anthropomorphic ghost, or simulator (e.g., with the body parts, or tissue being simulated).

Any of the various systems, devices, apparatuses, etc. in this disclosure may be sterilized (e.g., with heat, radiation, ethylene oxide, hydrogen peroxide) to ensure they are safe for use with patients, and the methods herein may comprise sterilization of the associated system, device, apparatus, etc.; e.g., with heat, radiation, ethylene oxide, hydrogen peroxide.

1. A method for determining a limit to a subject's autoregulation range, comprising:
determining blood flow values representative of a subject's blood flow in an autoregulation range of the subject during a period of time;
determining blood pressure values representative of the subject's blood pressure in the autoregulation range of the subject during the period of time;
determining a mathematical function that represents the blood flow values versus the blood pressure values within the autoregulation range;
using the mathematical function to produce a representation of the blood flow values versus the blood pressure values outside of the autoregulation range; and
predicting a limit to the autoregulation range using the representation of the blood flow values versus the blood pressure values outside of the autoregulation range.

2. The method of claim 1, wherein the predicted limit is a lower limit of autoregulation.

3. The method of claim 1, wherein the predicted limit is an upper limit of autoregulation.

4. The method of claim 1, wherein the step of determining blood flow values representative of the subject's blood flow in the autoregulation range of the subject during the period of time is performed non-invasively.

5. The method of claim 4, wherein the step of determining blood flow values representative of the subject's blood flow in the autoregulation range of the subject during the period of time is performed using a NIRS oximeter.

6. The method of claim 1, wherein the step of determining blood flow values representative of the subject's blood flow in the autoregulation range of the subject during the period of time includes determining a cerebral blood flow of the subject.

7. The method of claim 1, wherein the step of determining blood pressure values representative of the subject's blood pressure in the autoregulation range of the subject during the period of time includes determining mean arterial blood pressure (MAP) values of the subject or a parameter derived from the MAP values.

8. The method of claim 1, wherein the step of determining the mathematical function includes fitting a third degree polynomial equation to the blood flow values versus the blood pressure values within the subject's autoregulation range.

9. The method of claim 8, wherein the step of using the mathematical function to produce a representation of the blood flow values versus the blood pressure values outside of the autoregulation range includes extrapolating the third degree polynomial equation to represent the blood flow values versus the blood pressure values outside of the autoregulation range.

10. The method of claim 1, wherein the step of determining the mathematical function includes fitting one or more sigmoid functions to the blood flow values versus the blood pressure values within the subject's autoregulation range.

11. The method of claim 10, wherein the step of using the mathematical function to produce a representation of the blood flow values versus the blood pressure values outside of the autoregulation range includes extrapolating the one or more sigmoid functions to represent the blood flow values versus the blood pressure values outside of the autoregulation range.

12. The method of claim 1, wherein the step of determining blood flow values representative of the subject's blood flow in the autoregulation range of the subject is performed continuously during the period of time; and
wherein the step of determining blood pressure values representative of the subject's blood pressure flow in the autoregulation range of the subject is performed continuously during the period of time; and
wherein the step of predicting the limit to the autoregulation range using the representation of the blood flow values versus the blood pressure values outside of the autoregulation range is performed in real time.

13. A system configured to determine a limit to a subject's autoregulation range, comprising:
a blood flow measurement device configured to determine blood flow values representative of a subject's blood flow in an autoregulation range of the subject during a period of time and to produce first signals representative of the blood flow values;
a blood pressure sensing device configured to determine blood pressure values representative of the subject's blood pressure in the autoregulation range of the subject during the period of time and to produce second signals representative of the blood flow values; and
a system controller in communication with the blood flow measurement device and the blood pressure sensing device, the system controller including at least one processor and a memory device configured to store instructions, the stored instructions when executed cause the system controller to:
control the blood flow measurement device to determine the blood flow values representative of the subject's blood flow in the autoregulation range of the subject during the period of time and to produce the first signals representative of the blood flow values;
control the blood pressure sensing device to determine the blood pressure values representative of the subject's blood pressure in the autoregulation range of the subject during the period of time and to produce the second signals representative of the blood pressure values;
determine a mathematical function that represents the blood flow values versus the blood pressure values within the autoregulation range using the first signals and the second signals;
produce a representation of the blood flow values versus the blood pressure values outside of the autoregulation range using the mathematical function; and
predict a limit to the autoregulation range using the representation of the blood flow values versus the blood pressure values outside of the autoregulation range.

14. The system of claim 13, wherein the predicted limit is at least one of a lower limit of autoregulation or an upper limit of autoregulation.

15. The system of claim 13, wherein the blood flow measurement device is a NIRS oximeter configured to determine blood flow values non-invasively.

16. The system of claim 13, wherein the blood pressure sensing device is configured to determine mean arterial blood pressure (MAP) values of the subject or a parameter derived from the MAP values.

17. The system of claim 13, wherein the mathematical function is a third degree polynomial equation fit to the blood flow values versus the blood pressure values within the subject's autoregulation range, and the instructions that when executed cause the system controller to extrapolate the third degree polynomial equation to represent the blood flow values versus the blood pressure values outside of the autoregulation range.

18. The system of claim 13, wherein the mathematical function is a sigmoid function fit to the blood flow values versus the blood pressure values within the subject's autoregulation range, and the instructions that when executed cause the system controller to extrapolate the sigmoid function to represent the blood flow values versus the blood pressure values outside of the autoregulation range.

19. The system of claim 13, wherein the instructions that when executed cause the system controller to:
control the blood flow measurement device to continuously determine the blood flow values representative of the subject's blood flow in the autoregulation range during the period of time; and
control the blood pressure sensing device to continuously determine the blood pressure values representative of the subject's blood pressure in the autoregulation range during the period of time; and
predict the limit to the autoregulation range using the representation of the blood flow values versus the blood pressure values outside of the autoregulation range in real time.

20. (canceled)

21. A non-transitory computer readable medium storing executable instructions that when executed cause at least one processor to:
control a blood flow measurement device to determine blood flow values representative of the subject's blood flow in an autoregulation range of the subject during a period of time and to produce first signals representative of the blood flow values;
control a blood pressure sensing device to determine blood pressure values representative of the subject's blood pressure in the autoregulation range of the subject during the period of time and to produce second signals representative of the blood pressure values;
determine a mathematical function that represents the blood flow values versus the blood pressure values within the autoregulation range using the first signals and the second signals;
produce a representation of the blood flow values versus the blood pressure values outside of the autoregulation range using the mathematical function; and
predict a limit to the autoregulation range using the representation of the blood flow values versus the blood pressure values outside of the autoregulation range.
